(19) United States
(12) Patent Application Publication
(10) Pub. No.: US 2007/0179625 A1
(43) Pub. Date: Aug. 2, 2007

(54) Shoulder prosthesis and a system for implanting a shoulder prosthesis

(76) Inventors: Carl Ekholm, Onsala (SE); Anders Jonsson, Onsala (SE)

Correspondence Address: SCHWEGMAN, LUNDBERG, WOESSNER & KLUTH, P.A., P.O. BOX 2938, MINNEAPOLIS, MN, 55402, US

(21) Appl. No.: 10/593,940

(22) PCT Filed: Mar. 22, 2005
(86) PCT No.: PCT/SE05/00417

§ 371 Date: Sep. 22, 2006

(30) Foreign Application Priority Data
Mar. 23, 2004 (SE) 0400756-3

(51) Int. Cl. A61F 2/40; A61F 2/46

(52) U.S. Cl. 623/19.14; 623/23.27

(57) ABSTRACT

Shoulder prosthesis, including a stem provided to be inserted into a canal of the humerus shaft, a neck and a head supported by the neck. The neck is provided with two holes (1, 2) for anchoring screws (1a, 2a), namely a first hole (1) for a lateral anchoring screw (1a) and a second hole (2) for an anterior anchoring screw (2a). The lateral anchoring screw is provided to attach a first tubercle laterally to the neck and the anterior anchoring screw is provided to attach a second tubercle anterior to the neck. A system for implanting a shoulder prosthesis, comprises a targeting arm attachable to the neck of the prosthesis and is provided with guide means for guiding anchoring screws (1a, 2a) to be screwed into at least a lateral hole (1) and an anterior hole (2) in the neck. The targeting arm is also provided for holding a trial prosthesis.

### TECHNICAL FIELD

The present invention relates to a shoulder prosthesis and a system for implanting a shoulder prosthesis according to the preamble of claims 1 and 5 respectively.

1. Background Art

Replacement is undertaken in fresh fractures when functional repair of the humeral head is deemed impossible. The goal is of course to make the shoulder pain free and to reconstruct the joint for restored range of motion. The similar approach is standard procedure for femoral neck fractures and produces excellent results. Although shoulder hemi replacement arthroplasty works well with the rheumatoid and arthritic shoulder, humeral head replacement in the fracture situation has been less successful particularly regarding the range of motion obtained. The reason for this may not be perfectly clear but several explanations can be put forward.
In the fractured shoulder anatomy is distorted and landmarks may be lost. It may therefore be difficult to judge exactly how the humeral prosthesis should be fixed regarding height, retroversion, posterior offset etc.
The trauma situation involves not only skeletal parts but also shoulder soft tissue, particularly the rotator cuff. In addition the trauma may have caused injury to the innervation of, most commonly, the deltoid muscle. The fractures in which shoulder arthroplasty are considered are the severely comminute ones bearing relation to a more extensive soft tissue injury.
Of outmost importance for shoulder function is the function of the rotator cuff. This group of muscles insert at the lesser and greater tubercles. In the acute fracture situation both these tubercles have been avulsed. With less than perfect reduction of the tubercles the tension in the rotator cuff tendons will be altered, the direction of pull of the muscles may be changed. Furthermore, misplaced tubercles will cause impingement with the acromion or the edge of the glenoid joint surface.
Weak or unreliable fixation of the tubercles will prevent early active motion which in turn will lead to inferior range of motion.

2. Prior Art

Today a number of shoulder implants exist. They are fairly similar in design and basically developed for replacement of the arthritic shoulder. An example of a prior known shoulder prosthesis is shown in FIG. 1 inserted into the humeral shaft 20 and showing how the tubercles 21, 22 are to be attached. That prosthesis is further disclosed in U.S. Pat. No. 6,494,913.

With existing designs problems still exist re the insertion of the tubercles. Tubercles are reattached by sutures in the junction between the tubercle itself and the corresponding tendon to the laterally placed fins. In addition sutures between the tendons and drill holes in the proximal humeral stem are used. None of these sutures attach the tubercle at their normal centre of pull—hence the tubercles will not become fully stabilized. On rotation of the humerus the pull of the tendons will tend to lift the tubercles off the prosthetic neck, since the sutures are hinged at their attachment points laterally.

### DISCLOSURE OF INVENTION

The object of the present invention is to remove the problems associated with prior art prostheses.

Said objects are achieved by means of the shoulder prosthesis and the system for implanting the shoulder prosthesis whose characteristics are apparent from the accompanying claims 1 and 5 respectively.

### BRIEF DESCRIPTION OF DRAWINGS

The present invention will be further described in an embodiment with reference to the accompanying drawings, in which

FIG. 2 is a lateral view of a prosthesis according to the present invention positioned in the humerus, also showing an adjustable ring, forming part of the system according to the present invention

FIG. 3 is a schematic lateral view of the top of the prosthesis,

FIG. 4 is a top end view of the prosthesis and a targeting arm, forming part of the system according to the present invention.

### MODE FOR CARRYING OUT THE INVENTION

The main parts of the shoulder prosthesis and the system for implanting a shoulder prosthesis into a human being according to the present invention are as follows:
hole 1 for lateral anchoring screw 1a (directed slightly distally to leave room for anterior hole)
hole 2 for anterior anchoring screw 2a
hole 3 for targeting arm 10 attachment
hole 4 in anterior fin, also accepting targeting arm stabilizing peg
lateral fin 5
male part 6a of Morse taper 6
suture hole 7a at medial neck 7
prosthesis top end 8 (lateral view) being tapered
etch marks 9
targeting arm 10
peg 11 of targeting arm
sleeve 12 for guiding lateral screw
sleeve 13 for guiding anterior screw
adjustable ring 14 supported by the locking screw against the fracture
guide wire 15 for posterior cannulated screw 16a
hole 16 for posterior screw 16a

The principal aspects of the system according to the present invention are as follows.
A way of temporarily fixing a trial stem to the humeral shaft 20. This is done in such a way that adjustments could be made of the insertion depth i.e. the humeral height. The insertion device, the targeting arm 10, will be used for this purpose. The arm holding the trial stem is to be fixed to the humeral shaft by pins or similar and incorporate a height adjustment screw.
The targeting arm is coupled to the trial stem in such a way that it does not interfere with trial reduction of the tubercles and balancing of the rotator cuff. Therefore it will be attached between the junction of the lesser and greater tubercle, i.e. at an angle of about 45 degrees anterior to the lateral fin (tubercles are normally separated in this region).
Re-attachment of the tubercles will be improved relative to prior art. These are today normally stitched back, using heavy sutures, to the holes in the fin or through drill holes in the proximal humeral shaft. These sutures are passed through the tubercles themselves or through the corresponding rotator cuff tendon.

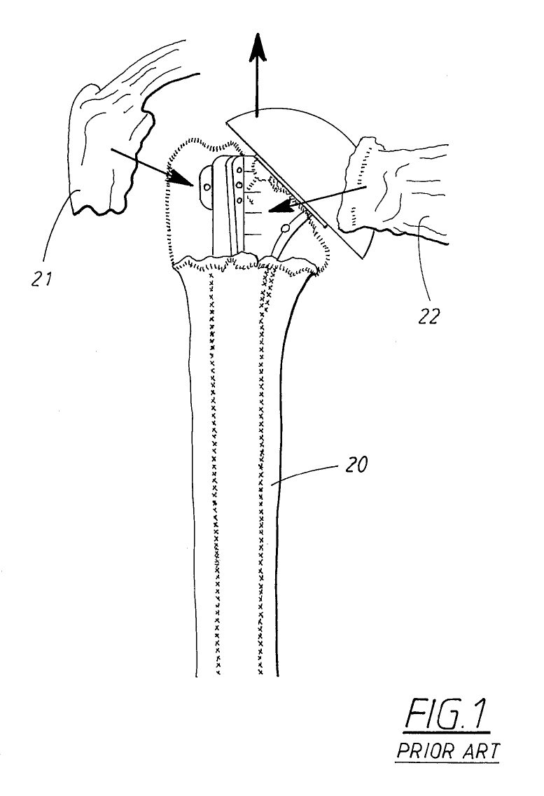

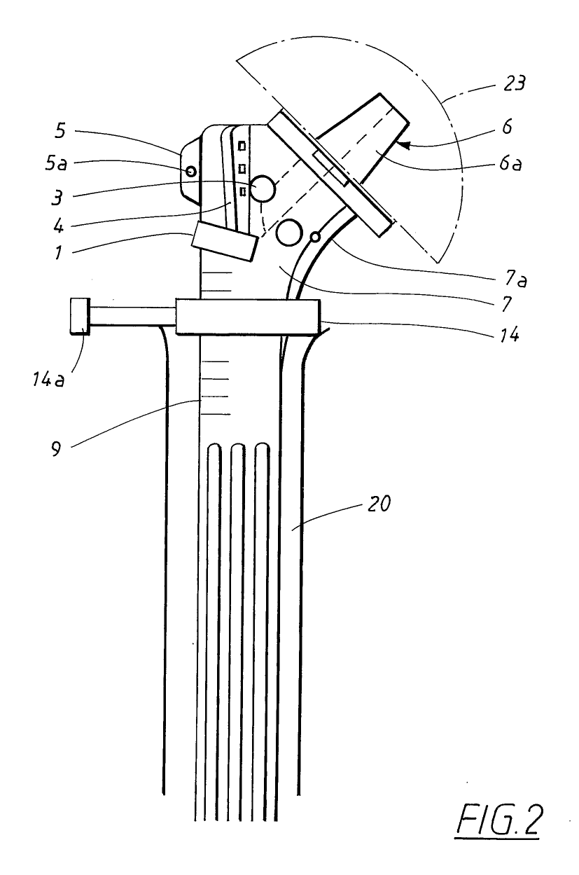

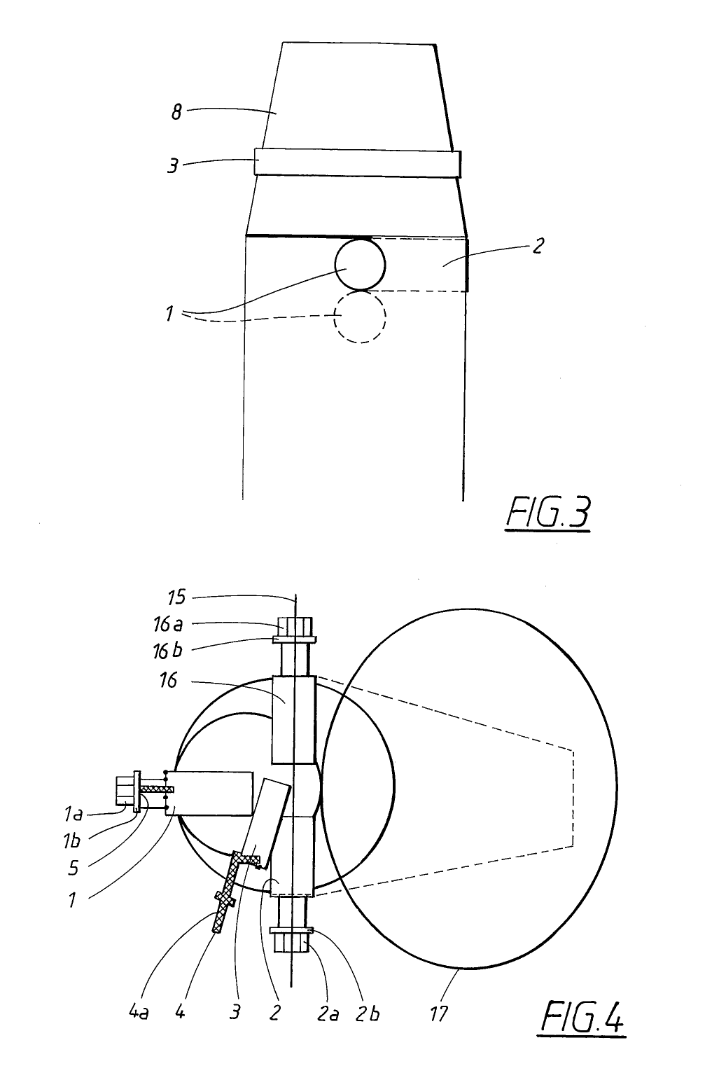

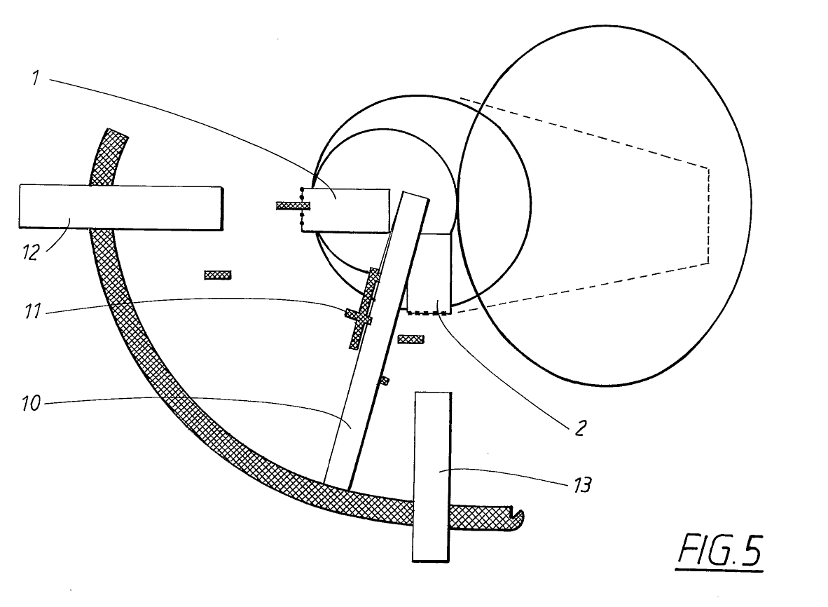

By means of the present invention it is enabled to
hold the tubercles tight to prosthesis and in contact with the humeral shaft in order to promote bony union without impingement
convey the forces of the rotator cuff to the humeral shaft
immobilize the tubercles well enough for early, pain free rehabilitation.
The use of screw fixation of the tubercles will improve the quality of the fixation.
   Two screws 1a, 2a one for each tubercle (i.e. lateral and anterior) would probably suffice.
   These screws, being angularly stable, will offer stable point of fixation.
   In conjunction with washers 1b, 2b they permit compression of the tubercles against the neck of the prostheses thereby obviating the need for removal of valuable bone from the tubercles to make the fit without impingement.
   The screws will also serve as secure anchoring points for the additional sutures that are needed.
   The screws are guided to their sites by the drill sleeves 12, 13 supported by the targeting arm 10.
Top end 8 of prosthetic stem tapered to give more room for tubercle re-insertion
Lateral fin 5 very short, not to encrouch on room for major tubercle
Anterior fin angled to point at bicipital groove. Thus it will be positioned at the junction between the lesser and the greater tubercle and allow full reduction of each of these tubercles
Tubercle fixation is accomplished by means of the two screws 1b, 2b. These screws are placed centrally in each tubercle in order to optimally engage the forces generated by the muscles acting on each tubercle.
   These screws are finely (machine) threaded and lock into corresponding holes 1, 2 in the neck of the prosthesis.
   They are angularly stable either by being screwed home to the bottom of their respective holes or by means of a compression sleeve.
   If a posterior screw 16 as shown in FIG. 4 is needed this may be cannulated and guided into place by the guide wire 15 inserted from anterior through the recipient hole.
   These screws 1a, 2a, 16a may also serve as the attachment point for sutures further anchoring the rotator cuff tendons.
   They will also allow the use of washers 1b, 2b, 16b to gain compression of the tubercle against the prosthetic stem and may thus minimize the need for “trimming” the interior of the tubercles.
      These washers may have different sizes and shapes to be optimized for each fracture.
The anchoring screws 1a, 2a, 16a are guided to their respective holes by means of the targeting arm 10 attached to the prosthetic neck at the junction with the anterior fin. It will thus only minimally encrouch on the room meant for the lesser tubercle.
   The targeting arm is screwed or bolted to a hole in the prosthetic neck and stabilized by engaging a small peg into the holes of the anterior fin.
   The targeting arm may also serve as device for holding the trial prosthesis during trial phase of the operation.
For further anchoring of the tubercles sutures may be attached to the holes 5a, 4a in the lateral and anterior fins 5, 4 and at the medial neck.
The prosthetic head 23 indicated in FIG. 2 by means of dashed and dotted lines is attached to the neck 7 by a Morse taper 6; the male part 6a sitting on the neck of the prosthesis and a female part sitting on the head.
Trial prosthesis should be modular, i.e. the one neck component can be easily fitted to stem components of different sizes.
The adjustable ring 14 locked to the stem, by means of a screw 14a, at chosen height, will enable the trial implant to be rested against edge of the fractured humerus 20 for assessment of suitable height. Readings can be made against the etch marks 9 in the trial prostheses corresponding to marks in the implant.

With the abovementioned prosthesis and system we expect to achieve
Improved positioning of the prosthesis
Improved balancing of the rotator cuff
Improved fixation of the rotator cuff
Early initiation of rehabilitation
Less initial pain
Shorter operating times

The present invention is not limited to the embodiment as described above and as shown in the accompanying drawings, but can be modified within the scope of the accompanying claims.

1. Shoulder prosthesis, including a stem provided to be inserted into a canal of the humerus shaft (20), a neck (7) and a head (23) supported by the neck, characterized therein that the neck (7) is provided with at least two holes (1, 2) for anchoring screws (1a, 2a), namely a first hole (1) for a lateral anchoring screw (1a) and a second hole (2) for an anterior anchoring screw (2a), said lateral anchoring screw being provided to attach a first tubercle laterally to the neck and said anterior anchoring screw being provided to attach a second tubercle anterior to the neck.

2. Shoulder prosthesis according to claim 1, characterized therein, that a third hole (16) in the neck (7) is provided for a posterior anchoring screw (16a) being provided to attach a third tubercle posteriorly to the neck of the prosthesis.

3. Shoulder prosthesis according to claim 1, characterized therein, that the top end (8) of the prosthetic stem is tapered.

4. Shoulder prosthesis according to claim 1, characterized therein, that a lateral fin (5) and a anterior fin (4) project from the neck (7) of the prosthesis, said lateral fin having a shorter length than the anterior fin, which is angled to point at bicipital groove.

5. System for implanting a shoulder prosthesis, characterized in a targeting arm (10) attachable to the neck (7) of the prosthesis and provided with guide means (12, 13) for guiding anchoring screws (1a, 2a) to be screwed into at least a lateral hole (1) and an anterior hole (2) in the neck, said targeting arm also being provided for holding the trial prosthesis.

6. System according to claim 3, characterized therein, that a further hole (3) is provided in the neck (7) and able to receive an attachment part of the targeting arm (10).

7. System according to claim 6, characterized therein, that the targeting arm (10) being stabilized by engaging a peg into a hole of anterior fin (4).

8. System according to claim 5, characterized therein, that a guide wire (15) is arranged for guiding a posterior screw (16) into place into a third hole (16) in the neck (7) of the prosthesis, said guide wire being inserted from anterior through the recipient hole.

9. System according to claim 5 including a trial stem provided with marks (9) for determining the depth of the stem in a canal of the humerus, characterized in a locking ring (14) surrounding the trial stem and resting on the end of the humeral shaft and able to lock the stem to a chosen depth.
